(19) United States
(12) Patent Application Publication
(10) Pub. No.: US 2025/0059544 A1
(43) Pub. Date: Feb. 20, 2025

(54) ANTISENSE OLIGONUCLEOTIDES (ASOS) THAT SUPPRESS SARS-COV-2 REPLICATION

(71) Applicant: The Children's Medical Center Corporation, Boston, MA (US)

(72) Inventors: Hao Wu, Brookline, MA (US); Longfei Wang, Boston, MA (US); Setu M. Vora, Brighton, MA (US)

(73) Assignee: The Children's Medical Center Corporation, Boston, MA (US)

(21) Appl. No.: 18/720,551

(22) PCT Filed: Dec. 16, 2022
(86) PCT No.: PCT/US2022/081796

§ 371 Date: Jun. 14, 2024

Related U.S. Application Data
(60) Provisional application No. 63/290,720, filed on Dec. 17, 2021.

(51) Int. Cl. C12N 15/113; A61P 31/14

(52) U.S. Cl. CPC C12N 15/1131; A61P 31/14; C12N 2310/11; C12N 2310/316; C12N 2310/3231

(57) ABSTRACT

Provided herein are antisense oligonucleotides for use in targeting SARS-CoV-2. Also provided herein are compositions comprising such oligonucleotides and methods for administering the oligonucleotides or compositions thereof to a subject for the purpose of treating or preventing a SARS-CoV-2 infection.

### RELATED APPLICATION

This application claims the benefit under 35 U.S.C. § 119(e) to U.S. Provisional Application No. 63/290,720, entitled “ANTISENSE OLIGONUCLEOTIDES (ASOS) THAT SUPPRESS SARS-COV-2 REPLICATION,” filed on Dec. 17, 2021, the entire contents of which are incorporated herein by reference.

### REFERENCE TO AN ELECTRONIC SEQUENCE LISTING

The contents of the electronic sequence listing (C123370229WO00-SEQ-RE.xml; Size: 14,736 bytes; and Date of Creation: Dec. 16, 2022) is herein incorporated by reference in its entirety.

### BACKGROUND

The coronavirus disease 2019 (COVID-19) pandemic caused by the beta coronavirus SARS-CoV-2 and its variants has taken a huge toll on human health globally. Despite the successful rollout of vaccines targeting SARS-CoV-2, effective therapies are still needed for treating those already infected by the virus or its variants. Upon entering a host cell, SARS-CoV-2 uses specialized proteins to inhibit host immunity and optimize virus production by reducing the translation of host proteins and enhancing the degradation of host mRNAs. The 5′ untranslated region of SARS-CoV-2 mRNA has been implicated in protecting the viral mRNA from this suppression and may present a novel therapeutic target.

### SUMMARY

Some aspects of the present disclosure relate to an oligonucleotide comprising a region of complementarity to a severe acute respiratory syndrome coronavirus 2 (SARS-CoV-2) 5′ untranslated region (UTR) target. In some embodiments, the oligonucleotide comprises a region of complementarity to the sequence of SARS-CoV-2 stem loop 1 (SL1), set forth as the sequence of SEQ ID NO: 1. In some embodiments, the region of complementarity is at least 17 nucleotides in length.

In some embodiments, the oligonucleotide comprises one more ribonucleosides and/or one or more deoxyribonucleosides. In some embodiments, at least 40% of the nucleotides in the oligonucleotide are ribonucleosides. In some embodiments, at least 40% of the nucleotides in the oligonucleotide are deoxyribonucleosides.

In some embodiments, at least one of the one or more ribonucleotides is a modified ribonucleoside. In some embodiments, the modified ribonucleoside is a locked nucleic acid (LNA)-modified ribonucleoside. In some embodiments, at least 40% of nucleosides in the oligonucleotide are LNA-modified ribonucleosides.

In some embodiments, the oligonucleotide comprises one or more modified internucleoside linkages. In some embodiments, the oligonucleotide comprises one or more phosphorothioate internucleoside linkages. In some embodiments, every internucleoside linkage is a phosphorothioate internucleoside linkage.

In some embodiments, the oligonucleotide comprises at least 12 consecutive nucleotides of the nucleotide sequence set forth by any one of SEQ ID NO: 2, SEQ ID NO: 3, or SEQ ID NO: 4. In some embodiments, the oligonucleotide comprises the nucleotide sequence of any one of SEQ ID NO: 2, SEQ ID NO: 3, or SEQ ID NO: 4.

In some embodiments, the oligonucleotide comprises the nucleotide sequence set forth as any one of the following, where “dN” represents a deoxyribonucleoside; “+N” represents a locked nucleic acid-modified ribonucleoside; and all nucleotides are linked by phosphodiester internucleoside linkages:

(SEQ ID NO: 2)
dC+CdTdG+G+GdA+AdGdG+TdA+TdAdA+AdC+CdTdT+TdA+AdT;
(SEQ ID NO: 3)
dG+GdTdT+T+GdTdTdA+CdC+T+G+G+GdA+A+G+G;
or
(SEQ ID NO: 4)
dGdT+TdA+C+C+T+G+G+GdAdAdGdG+TdA+TdA+AdA+C+
CdTdT+TdA+AdT.

In some embodiments, the oligonucleotide is not a nucleotide set forth by any one of SEQ ID NO: 5, SEQ ID NO: 6, or SEQ ID NO: 7.

Further aspects of the present disclosure relate to compositions comprising an oligonucleotide comprising a region of complementarity to a SARS-CoV-2 5′ UTR target. In some embodiments, the composition comprises an oligonucleotide comprising a region of complementarity to the sequence of SARS-CoV-2 SL1, set forth as the sequence of SEQ ID NO: 1. In some embodiments, the composition further comprises a pharmacologically acceptable excipient. In some embodiments, the composition further comprises a liposome or a nanoparticle.

Further aspects of the present disclosure relate to methods for treating a SARS-CoV-2 infection in a subject in need thereof or for reducing the risk of a SARS-CoV-2 infection in a subject in need thereof, such a method comprising administering to the subject an effective amount of an oligonucleotide comprising a region of complementarity to a SARS-CoV-2 5′ UTR target. In some embodiments, the method comprises administering to the subject an effective amount of an oligonucleotide comprising a region of complementarity to the sequence of SARS-CoV-2 SL1, set forth as the sequence of SEQ ID NO: 1.

In some embodiments, the administration reduces the translation of SARS-CoV-2 mRNA in cells of the subject. In some embodiments, the administration increases the degradation of SARS-CoV-2 mRNA in cells of the subject. In some embodiments, the administration reduces the production of SARS-CoV-2 viral particles in the subject.

In some embodiments, the subject is a mammal. In some embodiments, the subject is a human. In some embodiments, the subject is a human neonate, a human infant, a human adult, or an elderly human. In some embodiments, the administration occurs when the subject is more than 65 years of age. In some embodiments, the subject is immune-senescent, immune-compromised, is infected with human immunodeficiency virus (HIV), has chronic lung disease, asthma, cardiovascular disease, cancer, a metabolic disorder, chronic kidney disease, liver disease, is malnourished, or is frail.

In some embodiments, the subject is a companion animal, a research animal, or a domesticated animal. In some embodiments, the subject is an adult or elderly companion animal.

In some embodiments, the administration is systemic. In some embodiments, the administration is intravenous, intramuscular, oral, sublingual, or inhaled. In some embodiments, the administration occurs more than once. In some embodiments, the administration is prophylactic.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1G. SARS-CoV-2 5′ UTR bypasses Nsp1-mediated inhibition of translation. FIG. 1A: Schematic of translational reporters. 5′ UTR sequences from control, MAVs, or SARS-CoV-2 were placed upstream of the mScarlet reporter (left panel). MBP or MBP-Nsp1 (right panel) were both downstream of control 5′ UTR and were co-transfected along with each reporter plasmid. A CMV promoter was used to drive expression in all constructs. FIG. 1B: Representative images of HeLa cells co-transfected with control 5′UTR reporter or SARS-CoV-2 5′ UTR reporter and either MBP alone or MBP-Nsp1 and visualized for DNA by Hoechst, MBP by indirect immunofluorescence, and mScarlet by in situ fluorescence. Successfully transfected cells difficult to visualize due to low intensity are outlined here and in other figures. FIG. 1C: Quantification of relative mScarlet intensity of data corresponding to FIG. 1B. FIG. 1D: Representative images of HeLa cells transfected with MAVs 5′ UTR reporter. FIG. 1E: Quantification of relative mScarlet intensity in FIG. 1D.

FIG. 1F: HeLa cells transfected with either ORF3a-GFP (left panel) or ORF8-GFP (right panel) downstream of SARS-CoV-2 5′ UTR. FIG. 1G: Quantification of relative GFP intensity in FIG. 1F. Error bars correspond to standard error of the mean except where otherwise noted. Scale bars are shown in each bottom right image and correspond to 10 microns (μm).

FIGS. 2A-2C. The SL1 stem-loop of the 5′ UTR is necessary and sufficient for evasion of Nsp1-mediated translation suppression. FIG. 2A: Schematic representation of 5′ UTR, SL1 5′ UTR and ASL1 5′ UTR placed upstream of mScarlet (top panel), and of SARS-CoV-2 leader sequence containing SL1 along with its incorporation into subgenomic RNAs (SEQ ID NO:8) (bottom panel). FIG. 2B: Representative images of HeLa cells co-transfected with SARS-CoV-2 5′ UTR reporter and either MBP alone or MBP-Nsp1, and visualized for DNA by Hoechst and mScarlet by in situ fluorescence. FIG. 2C: Quantification of relative mScarlet intensity of data corresponding to FIG. 2B.

FIGS. 3A-3F. Nsp1 N- and C-terminal domains cooperate to drive viral translation selectivity. FIG. 3A: Schematic of co-expression system with CoV-2 or control reporter along with various fragments of Nsp1 (FL, NT, CT, NT+CT) or extended linker mutants (linker1, linker2). FIG. 3B: mScarlet fluorescence intensity in HeLa cells co-transfected with either the CoV-2 (top row) or control reporter (bottom row) along with various mutants of Nsp1. FIG. 3C: Quantification of fluorescent intensity in FIG. 3B of CoV-2 (left) or control reporters (middle) and the ratio of CoV-2/Control (right) with different Nsp1 mutants. Dashed line marks ratio of 1. FIG. 3D: mScarlet fluorescence intensity in 293T cells as in FIG. 3B. FIG. 3E: Quantification of FIG. 3D. FIG. 3F: Relative luciferase activity in 293T cells assay co-transfected with CoV-2 firefly luciferase and control renilla luciferase reporters along with various Nsp1 mutants. The ratio of Firefly/Renilla luciferase was normalized and plotted in 3 replicates. Scale bars for panels FIG. 3B and FIG. 3D both correspond to 10 microns. Error bars represent standard deviation.

FIGS. 4A-4E. ASOs targeting SL1 renders the SARS-CoV-2 5′ UTR susceptible to Nsp1-mediated shutdown. FIG. 4A: Schematic of SL1 region and various ASOs (which are all LNA mixmers unless otherwise noted) (SEQ ID NO: 2-4, and 9-12). FIG. 4B: Initial screen of ASO activity. Each ASO was transfected at 50 nM with CoV-2 reporter along with either MBP alone or MBP-Nsp1. Bar on far right indicates co-transfection with a reporter lacking SL1 as a control. FIG. 4C: Images of HeLa cells transfected with 50 nM AS04, 7, 6, or a control ASO along with CoV-2 or ASL1 reporter and either MBP or MBP-Nsp1. FIG. 4D: Quantification of FIG. 4C. FIG. 4E: Dose-response assay of each ASO. Cells were transfected with CoV-2 reporter and MBP or MBP-Nsp1 as above. ASOs were transfected at either 25, 50, or 100 nM.

FIGS. 5A-5F. ASOs targeting SL1 produce stable loss of function to inhibit SARS-CoV-2 replication in vitro and ASO4 provides significant protection against SARS-CoV-2-induced lethality in K18-hACE2 mice. FIG. 5A: Various ASOs along with CoV-2 reporter and MBP-Nsp1 were transiently transfected into Vero E6 cells and reporter intensity was measured daily over the course of 72 hours, shown for each ASO (control, AS04, and AS07) at each timepoint. Because expression from transfected plasmids naturally changes over time, each datapoint was normalized to intensity of a parallel control where no ASO was included. FIG. 5B: Percent of successfully transfected cells (marked by mScarlet positivity (mSc+)) that were nucleocapsid positive (N+) by ASO treatment at various multiplicities of infection (MOIs). Error bars represent standard deviation. FIG. 5C: Nucleocapsid intensity plotted against mSc obtained by flow cytometry for each treatment (infected at MOI 0.5). Quadrants demarcate mSc+, N+ cells (top right quadrant), and the corresponding percentage of cells is listed in each corner. FIG. 5D: Schematic for mouse infection experiment. K18-hACE-2 mice were treated with daily intranasally administered Control ASO or AS04 for 4 days following infection with 2,500 PFU of SARS-CoV-2 and monitored for weight loss and survival for 14 days. FIG. 5E: Average percent weight loss over time for control ASO or AS04 after infection (left) and individual weight loss trajectories (right). FIG. 5F: Survival curves over time for control ASO or AS04 after infection.

FIGS. 6A-6B. Model for Nsp1-driven viral translation selectivity and its disruption via ASO targeting of the highly conserved SL1 region. FIG. 6A: Nsp1 shuts down host translation, mainly by blocking the mRNA entry channel of the 40S ribosome which ultimately results in host mRNA degradation. The SL1 region in SARS-CoV-2 5′UTR allows evasion of translational suppression, leading to selective viral translation. Targeting SL1 via ASO makes SARS-CoV-2 5′UTR vulnerable to Nsp1-mediated translation suppression, resulting in loss of translation of Nsp1 itself and restoration of host translation, allowing anti-viral defense to more effectively halt viral replication. FIG. 6B: Alignment of the AS04 target sequence with SL1 sequences from SARS-CoV-2 variants of concern showing complete conservation of the sequence targeted by AS04 (SEQ ID NO: 1, 13-14).

FIG. 7. SARS-CoV-2 5′ UTR secondary structure prediction. Schematic depicting predicted stem loops of the SARS-CoV-2 5′ UTR (SEQ ID NO: 15).

FIG. 8. Immunofluorescence of MBP-Nsp1 and in situ fluorescence of reporter activity in HeLa and 293T cells. Cells were stained and visualized for various Nsp1 fragments tagged with MBP. Reporter activity was visualized with in situ mScarlet intensity. In the NT+CT condition, both NT and CT were tagged with MBP. Cells were counterstained with Hoescht 33342 to visualize nuclei.

FIGS. 9A-9B. Nsp1^{R124A} and Nsp1^{ΔKSF} are defective in viral translation selectivity.

FIG. 9A: Images of reporter intensity in HeLa cells transfected with either CoV-2 or control reporter along with various mutants of Nsp1. FIG. 9B: Mean fluorescence intensity of CoV-2 reporter or control reporter when co-transfected with Nsp1, Nsp1R^{124A}, or Nsp1^{ΔKSF} (left and middle panels) and the ratio between the two reporters (right panel). Error bars represent standard deviation.

### DETAILED DESCRIPTION OF CERTAIN EMBODIMENTS

Some aspects of the present disclosure are based, at least in part, on the finding that antisense oligonucleotides targeting the 5′ untranslated region (UTR) of severe acute respiratory syndrome coronavirus 2 (SARS-CoV-2) mRNA transcripts, specifically stem loop 1 (SL1), may be used to reduce SARS-CoV-2 virulence by nullifying protection of SARS-CoV-2 transcripts from the SARS-CoV-2-mediated host translation block. As described herein, these antisense oligonucleotides may be used to treat or prevent SARS-CoV-2 infection in subjects by reducing translation of viral proteins and/or enhancing degradation of viral transcripts, thereby reducing overall production of virus particles. As further described, this approach is useful for the treatment or prevention of either the original SARS-CoV-2 virus or its subsequent variants, and may be combined with other methods for treating or reducing the risk for SARS-CoV-2 infection, such as vaccination against SARS-CoV-2. In some embodiments, the antisense oligonucleotides disclosed herein are useful for treating (both prophylactically and therapeutically) SARS-CoV-2 infection in vulnerable populations (e.g., neonates, elderly, immunosenescent, or immunocompromised individuals).

### Antisense Oligonucleotides

Without wishing to be bound by theory, during infection by the SARS-CoV-2 beta coronavirus, the positive-sense RNA viral genome is transcribed to produce transcripts encoding two partially overlapping open reading frames, ORF1a and ORF1b, which are subsequently translated and proteolytically cleaved by host cells to produce 16 mature non-structural proteins (numbered Nsp1 through Nsp16). These non-structural proteins include virulence factors which are suggested to perform a variety of functions, including suppression of host gene expression and immune responses (e.g., by repurposing host translation machinery to preferentially translate SARS-CoV-2 viral RNA transcripts and by preventing the induction of Type 1 interferons). SARS-CoV-2 Nsp1 in particular suppresses the translation of host mRNAs by directly binding to the mRNA entry channel of the 40S small ribosomal subunit via its C-terminal domain (CT). Additionally, Nsp1 reduces the total pool of cytosolic host mRNAs available to infected cells by promoting the degradation of host mRNAs and inhibiting their nuclear export. Mutations in Nsp1 that disrupt ribosome binding also abolish the effect on mRNA degradation, implying that the increased degradation of host mRNAs observed in infected cells is likely downstream of the translational block. Together these two mechanisms synergistically impair host protein expression in SARS-CoV-2 infected cells.

However, while SARS-CoV-2 effectively inhibits the translation of host proteins, Nsp1 evidently does not interfere with the translation of SARS-CoV-2 viral proteins. The 5′ UTR of SARS-CoV-2 viral RNA is critical for circumventing the translation block mediated by Nsp1. This region comprises a variety of stem loops, of which stem loop 1 (SL1) appears to be essential for bypassing the effect of Nsp1, perhaps by interaction with Nsp1 and/or the 40S small ribosomal subunit. Given the importance of SL1 for maintaining efficient translation of SARS-CoV-2 proteins in the presence of Nsp1, SARS-CoV-2 virulence and/or infectivity could potentially be reduced or eliminated by means of a SL1-binding agent, such as, for example, an oligonucleotide complementary to SL1. Such an agent would be of use for treating and/or preventing SARS-CoV-2 infection in subject populations, such as human populations, especially as the SL1 sequence is highly conserved between the original SARS-CoV-2 virus and its subsequent variants.

In some aspects, the present disclosure describes an oligonucleotide comprising a region of complementarity to the 5′ UTR of a SARS-CoV-2 viral RNA. In some embodiments, such an oligonucleotide is capable of interacting (e.g., through Watson-Crick base pairing) with a region of the 5′ UTR of a SARS-CoV-2 viral RNA. In some embodiments, an interaction between an oligonucleotide described herein and a region of the 5′ UTR of a SARS-CoV-2 viral RNA reduces the virulence and/or infectivity of SARS-CoV-2 in a subject. In some embodiments, an oligonucleotide contemplated herein is an antisense oligonucleotide.

In some embodiments, an oligonucleotide described herein comprises a region of complementary to SL1 in the 5′ UTR of a SARS-CoV-2 viral RNA. In some embodiments, an oligonucleotide described herein is complementary to part or all of a SARS-CoV-2 SL1 sequence. In some embodiments, an oligonucleotide described herein is further complementary to one or more additional nucleotides of the 5′ UTR of a SARS-CoV-2 viral RNA.

In some embodiments, an oligonucleotide described herein comprises a region of complementarity to part or all of the SL1 sequence of the originally discovered SARS-CoV-2 virus. In some embodiments, an oligonucleotide described herein comprises a region of complementarity to part or all of the SL1 sequence of a SARS-CoV-2 variant, such as a variant of concern (VOC) as identified by the United States Centers for Disease Control and Prevention (CDC), such as, but not limited to, B.1.1.7 (alpha), B.1.351 (beta), P.1 (gamma), B.1.617.2 (delta), B.1.427 and B.1.429 (epsilon), B.1.525 (eta), B.1.526 (iota), B.1.617.1 (kappa), B.1.1.529 (omicron), B.1.621 (mu), and P.2 (zeta) variant SARS-CoV-2. In some embodiments, an oligonucleotide described herein comprises a region of complementarity to part or all of the sequence set forth as: AUUAAAGGUUUAUACCUUCCCAGGUAACAAACC (SEQ ID NO: 1).

In some embodiments, an oligonucleotide described herein comprises a region of complementarity that is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 21 nucleotides in length, at least 22 nucleotides in length, at least 23 nucleotides in length, at least 24 nucleotides in length, at least 25 nucleotides in length, at least 26 nucleotides in length, at least 27 nucleotides in length, at least 28 nucleotides in length, at least 29 nucleotides in length, at least 30 nucleotides in length, at least 31 nucleotides in length, at least 32 nucleotides in length, or at least 33 nucleotides in length. In some embodiments, an oligonucleotide described herein comprises a region of complementarity is between 10 and 15 nucleotides in length, between 15 and 20 nucleotides in length, between 20 and 25 nucleotides in length, between 25 and 30 nucleotides in length, between 20 and 30 nucleotides in length, between 15 and 30 nucleotides in length, between 10 and 30 nucleotides in length, or between 10 and 33 nucleotides in length.

In some embodiments, an oligonucleotide described herein comprises a region of complementarity that is fully (i.e., 100%) complementary to a region of the 5′ UTR of a SARS-CoV-2 viral RNA, e.g., all or part of a SL1 sequence (SEQ ID NO: 1). In some embodiments, an oligonucleotide described herein comprises a region of complementarity that is partially (i.e., less than 100%) complementary to a region of the 5′ UTR of a SARS-CoV-2 viral RNA, e.g., all or part of a SL1 sequence (SEQ ID NO: 1). In some embodiments, an oligonucleotide described herein comprises a region of complementarity that is at least 80%, at least 90%, at least 95%, or at least 99% complementary to a region of the 5′ UTR of a SARS-CoV-2 viral RNA, e.g., all or part of a SL1 sequence (SEQ ID NO: 1). In some embodiments, an oligonucleotide described herein comprises a region of complementarity that contains 1, 2, or 3 mismatched nucleotides as compared to a region of the 5′ UTR of a SARS-CoV-2 viral RNA, e.g., all or part of a SL1 sequence (SEQ ID NO: 1), where each mismatched nucleotide comprises a different nucleobase (e.g., adenine (A), uracil (U), guanine (G), cytosine (C)) than that which is encoded by the 5′ UTR of a SARS-CoV-2 viral RNA at the corresponding position.

In some embodiments, an oligonucleotide described herein is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 21 nucleotides in length, at least 22 nucleotides in length, at least 23 nucleotides in length, at least 24 nucleotides in length, at least 25 nucleotides in length, at least 26 nucleotides in length, at least 27 nucleotides in length, at least 28 nucleotides in length, at least 29 nucleotides in length, at least 30 nucleotides in length, at least 31 nucleotides in length, at least 32 nucleotides in length, at least 33 nucleotides in length, at least 35 nucleotides in length, at least 40 nucleotides in length, at least 45 nucleotides in length, or at least 50 nucleotides in length. In some embodiments, an oligonucleotide described herein is between 10 and 15 nucleotides in length, between 15 and 20 nucleotides in length, between 20 and 25 nucleotides in length, between 25 and 30 nucleotides in length, between 20 and 30 nucleotides in length, between 15 and 30 nucleotides in length, between 10 and 30 nucleotides in length, between 10 and 33 nucleotides in length, between 10 and 40 nucleotides in length, or between 10 and 50 nucleotides in length.

In some embodiments, an oligonucleotide described herein comprises one or more ribonucleosides. In some embodiments, an oligonucleotide described herein comprises one or more deoxyribonucleosides. In some embodiments, an oligonucleotide described herein comprises one or more ribonucleosides and one or more deoxyribonucleosides. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of nucleotides in an oligonucleotide described herein are ribonucleosides. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of nucleotides in an oligonucleotide described herein are deoxyribonucleosides.

In some embodiments, at least one of the one or more ribonucleosides in an oligonucleotide described herein are modified ribonucleosides. In some embodiments, at least one of the one or more deoxyribonucleosides in an oligonucleotide described herein are modified deoxyribonucleosides. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of nucleotides in an oligonucleotide described herein are modified ribonucleosides or modified deoxyribonucleosides. In some embodiments, every nucleotide in an oligonucleotide described herein are modified ribonucleosides or modified deoxyribonucleosides. As defined herein, a modified nucleoside is a nucleoside comprising a sugar (ribose) and/or a nucleobase modification, where such a modification is not naturally occurring or does not occur naturally in nucleosides of a particular species (subject). Modified nucleosides include, for example, ribonucleosides and deoxyribonucleosides comprising a sugar (ribose) with an additional moiety at one or more carbon positions. Examples of modified nucleosides include, for example, 2′-nucleosides, such as 2′-deoxy, 2′-O-methyl (2′-O-Me), 2′-O-methoxyethyl (2′-MOE), locked nucleic acids (LNA, i.e., methylene-bridged nucleic acids), ethylene-bridged nucleic acids (ENA), and (S)-constrained ethyl-bridged nucleic acids (cEt). Other alternative modified nucleosides are well known the relevant art. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of nucleotides in an oligonucleotide described herein are LNA-modified ribonucleosides.

In some embodiments, one or more internucleoside linkages connecting nucleosides of an oligonucleotide described herein are modified internucleoside linkages. In some embodiments, every internucleoside linkage of an oligonucleotide described herein are modified internucleoside linkages. As defined herein, a modified nucleoside linkage is a nucleoside linkage that is not naturally occurring or does not occur naturally between nucleosides of a particular species (subject). In some embodiments, a modified internucleoside linkage is an internucleoside linkage that is not a phosphodiester internucleoside linkage. Examples of modified internucleoside linkages include, for example, phosphorothioate internucleoside linkages. Other alternative modified internucleoside linkages are well known the relevant art. In some embodiments, one or more internucleoside linkages connecting nucleosides of an oligonucleotide described herein are phosphorothioate internucleoside linkages. In some embodiments, every internucleoside linkage of an oligonucleotide described herein are phosphorothioate internucleoside linkages.

In some embodiments, an oligonucleotide described herein comprises at least 12, at least 13, at least 14, at least 15, at least 16, at least 17, at least 18, at least 19, at least 20, at least 21, at least 22, at least 23, or at least 24 consecutive nucleotides of the nucleotide sequence set forth as:

(SEQ ID NO: 2)
CCTGGGAAGGTATAAACCTTTAAT.

In some embodiments, an oligonucleotide described herein comprises at least 12, at least 13, at least 14, at least 15, at least 16, at least 17, at least 18, or at least 19 consecutive nucleotides of the nucleotide sequence set forth as:

(SEQ ID NO: 3)
GGTTTGTTACCTGGGAAGG.

In some embodiments, an oligonucleotide described herein comprises at least 12, at least 13, at least 14, at least 15, at least 16, at least 17, at least 18, at least 19, at least 20, at least 21, at least 22, at least 23, at least 24, at least 25, at least 26, at least 27, or at least 28 consecutive nucleotides of the nucleotide sequence set forth as:

(SEQ ID NO: 4)
GTTACCTGGGAAGGTATAAACCTTTAAT.

In some embodiments, an oligonucleotide described herein comprises the nucleotide sequence of any one of SEQ ID NO: 2, SEQ ID NO: 3, or SEQ ID NO: 4. In some embodiments, an oligonucleotide described herein consists of the nucleotide sequence of any one of SEQ ID NO: 2, SEQ ID NO: 3, or SEQ ID NO: 4. In some embodiments, an oligonucleotide described herein does not consist of a nucleotide sequence set forth as any one of GAAAGTTGGTTGGTTT (SEQ ID NO: 5), GTTTGTTACCTGGGAA (SEQ ID NO: 6); or GTTACCTGGGAAGGT (SEQ ID NO: 7).

In some embodiments, an oligonucleotide described herein is a mixmer. As defined herein, a mixmer is an oligonucleotide comprising alternating LNA nucleosides and deoxyribonucleosides. Nucleosides of a mixmer may alternate between LNA and DNA in a regularly repeating pattern (e.g., alternating stretches of LNA and DNA are equal in length) or in a pattern that is not regularly repeating (e.g., alternating stretches of LNA and DNA are variable in length). In some embodiments, an oligonucleotide described herein is a mixmer comprising the modified nucleotides sequence set forth in any one of the following, where “dN” represents a deoxyribonucleoside; “+N” represents a locked nucleic acid-modified ribonucleoside; and nucleotides are linked by phosphodiester internucleoside linkages:

(SEQ ID NO: 2)
dC+CdTdG+G+GdA+AdGdG+TdA+TdAdA+AdC+CdTdT+
TdA+AdT;
(SEQ ID NO: 3)
dG+GdTdT+T+GdTdTdA+CdC+T+G+G+GdA+A+G+G;
or
(SEQ ID NO: 4)
dGdT+TdA+C+C+T+G+G+GdAdAdGdG+TdA+TdA+AdA+
C+CdTdT+TdA+AdT

### Compositions

In some embodiments, compositions (e.g., pharmaceutical compositions) of the present disclosure comprise an oligonucleotide described herein. As contemplated herein, the terms “composition” and “formulation” may be used interchangeably.

In some embodiments, a composition may comprise an oligonucleotide described herein and one or more pharmacologically acceptable excipients. A pharmacologically acceptable excipient may enhance stability of an oligonucleotide described herein, enhance delivery of the oligonucleotide to cells of a subject to which the composition is administered, permit sustained or delayed release of the oligonucleotide upon administration, alter the biodistribution of the oligonucleotide (e.g., target the oligonucleotide to specific tissues or cell types), or reduce host immunity against the oligonucleotide. Examples of pharmacologically acceptable excipients includes any and all solvents, dispersion media, diluents or other liquid vehicles, dispersion or suspension aids, surface active agents, isotonic agents, thickening or emulsifying agents, and preservatives, as are known in the art. In some embodiments, a pharmacologically acceptable excipient comprises an aqueous solution or buffer. In some embodiments, the composition is isotonic, relative to a biological fluid of a subject (i.e., blood) to which the composition is to be administered. In some embodiments, the composition has a pH between 7 and 8, or optimally a pH of about 7.4.

In some embodiments, a composition may comprise an oligonucleotide described herein and a liposome or a nanoparticle. In some embodiments, a liposome or nanoparticle added to such a composition enhances delivery of the oligonucleotide to cells of a subject to which the composition is administered.

Liposomes are artificially prepared vesicles which may primarily be composed of a lipid bilayer and may be used as a delivery vehicle for the administration of one or more compounds in a pharmaceutical composition. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain an acidic, basic, or neutral pH in order to improve the delivery of the pharmaceutical composition.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are incorporated herein by reference. Characteristics of liposomes for use in enhancing the delivery of antisense oligonucleotides are described, for example, in Gagliardi and Ashizawa “The Challenges and Strategies of Antisense Oligonucleotide Drug Delivery” Biomedicines 2021, 9, 433, the contents of which are incorporated herein by reference.

Nanoparticles are artificially prepared particles that may contain organic, inorganic, naturally occurring, and/or synthetic nanoscale materials (nanomaterials) and generally range in size from 20 nm to 1000 nm. Examples of materials used in nanoparticles for enhancing delivery of antisense oligonucleotides include, without limitation, poly d,l-lactide (PLA), d,l-lactide-co-glycolide (PLGA), polyethylene glycol (PEG), polyethylenimine (PEI), chitosan/pluronic F68-coated polyisobutylcyanoacrylate (PIBCA), silver nanoparticles, and calcium phosphate (CP)-based nanoparticles. Additional examples of nanoparticles for use in enhancing the delivery of antisense oligonucleotides are described, for example, in Falzarano, et al. “Nanoparticle delivery of antisense oligonucleotides and their application in the exon skipping strategy for Duchenne muscular dystrophy” Nucleic Acid Ther, 24(1), 87-100, the contents of which are incorporated herein by reference.

In some embodiments, liposomes and nanoparticles may be formulated for targeted delivery. As a non-limiting example, a liposome or nanoparticle may be formulated for targeted delivery to one or more specific organs, tissues, and/or cells, such as those in the respiratory tract (e.g., lungs) or nervous system (e.g., brain). Examples of liposomes used for targeted delivery include, but are not limited to, those described in and methods of making liposomes described in US Patent Publication No. US20130195967, the contents of which are incorporated herein by reference.

### Kits

Also encompassed by the present disclosure are kits (e.g., pharmaceutical packs). The kits provided may comprise a pharmaceutical composition or oligonucleotide described herein and a container (e.g., a vial, ampule, bottle, syringe, and/or dispenser package, or other container suitable for storage and/or administration). In some embodiments, provided kits may optionally further include a second container comprising a pharmaceutical excipient for dilution or suspension of a pharmaceutical composition or oligonucleotide described herein. In some embodiments, the pharmaceutical composition or oligonucleotide described herein provided in the first container and the second container are combined to form one dosage unit.

Thus, in one aspect, provided are kits including a first container comprising a compound or pharmaceutical composition described herein. In certain embodiments, the kits are useful for treating a disease (e.g., coronavirus disease 2019 (i.e., COVID-19)) in a subject in need thereof. In certain embodiments, the kits are useful for preventing a disease (e.g., COVID-19) in a subject in need thereof.

In certain embodiments, a kit described herein further includes instructions for using the pharmaceutical composition or oligonucleotide included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., COVID-19) in a subject in need thereof. In certain embodiments, the kits and instructions provide for preventing a disease (e.g., COVID-19) in a subject in need thereof. A kit described herein may include one or more additional pharmaceutical agents described herein as a separate composition.

### Administration of Antisense Oligonucleotides

The terms “treatment,” “treat,” and “treating” refer to reversing, alleviating, delaying the onset of, or inhibiting the progress of a disease (e.g., COVID-19) described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen or the likelihood for future exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease and is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of a compound (e.g., an oligonucleotide described herein), the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound (e.g., an oligonucleotide described herein) administered in a single dose. In some embodiments, an effective amount is the combined amounts of a compound (e.g., an oligonucleotide described herein) administered in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a composition or compound (e.g., an oligonucleotide described herein) in or on a subject. A composition or compound (e.g., an oligonucleotide described herein) may be administered systemically (e.g., via intravenous injection) or locally (e.g., via local injection). In some embodiments, the composition or compound (e.g., an oligonucleotide described herein) is administered orally, intravenously, topically, intranasally, or sublingually. Parenteral administrating is also contemplated. The term “parenteral” as used herein includes subcutaneous, intracutaneous, intravenous, intramuscular, intraarticular, intraarterial, intrasynovial, intrasternal, intrathecal, intralesional, intradermally, and intracranial injection or infusion techniques. In some embodiments, the administering is done intramuscularly, intradermally, orally, intravenously, topically, intranasally, intravaginally, or sublingually. In some embodiments, the composition or compound (e.g., an oligonucleotide described herein) is administered prophylactically.

In some embodiments, a composition or compound (e.g., an oligonucleotide described herein) is administered once or is administered repeatedly (e.g., 2, 3, 4, 5, or more times). For multiple administrations, the administrations may be done over a period of time (e.g., 6 months, a year, 2 years, 5 years, 10 years, or longer). In some embodiments, the composition or compound (e.g., an oligonucleotide described herein) is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later). In some embodiments, the composition or compound (e.g., an oligonucleotide described herein) is administered more than twice, or is administered until a subject is free of symptoms of a disease (e.g., COVID-19) or until the risk of developing the disease subsides.

In some embodiments, a composition or compound (e.g., an oligonucleotide described herein) is administered to a subject for the purpose of treating or preventing an infection by a SARS-CoV-2 virus. In some embodiments, a composition or compound (e.g., an oligonucleotide described herein) is administered to a subject for the purpose of treating or preventing an infection by the originally discovered SARS-CoV-2 virus. In some embodiments, a composition or compound (e.g., an oligonucleotide described herein) is administered to a subject for the purpose of treating or preventing an infection by a SARS-CoV-2 variant, such as a variant of concern (VOC) as identified by the United States Centers for Disease Control and Prevention (CDC), such as, but not limited to, B.1.1.7 (alpha), B.1.351 (beta), P.1 (gamma), B.1.617.2 (delta), B.1.427 and B.1.429 (epsilon), B.1.525 (eta), B.1.526 (iota), B.1.617.1 (kappa), B.1.1.529 (omicron), B.1.621 (mu), and P.2 (zeta) variant SARS-CoV-2.

As defined herein, a “subject” refers to a living organism to which administration is contemplated. In some embodiments, a subject is a mammal. In some embodiments, the subject is a non-human animal (e.g., primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey)). In some embodiments the subject is a domesticated animal (e.g., cattle, pig, horse, sheep, goat) or a companion animal (i.e., a pet or service animal, e.g., cat or dog). In some embodiments, the subject is a fish, reptile, or amphibian. The non-human animal may be a male or female at any stage of development. The non-human animal may be a transgenic animal or genetically engineered animal.

In some embodiments, the subject is a human. In some embodiments, the subject is a human infant. In some embodiments, the human infant is a neonate that is less than 28 days of age. In some embodiments, the human infant is less than 1, 1, 2, 3, 4, 5, 6, 7, 8, 9, 10, 11, 12, 13, 14, 15, 16, 17, 18, 19, 20, 21, 22, 23, 24, 25, 26, 27, or 28 days of age at the time of administration.

In some embodiments, the human subject is more than 28 days of age (e.g., 1 month, 2 months, 3 months, 4 months, 5 months, 6 months, 7 months, 8 months, 9 months, 10 months, 11 months, 12 months, 2 years, 3 years, 4 years, 5 years, 10 years, 11 years, 12 years, 13 years, 14 years, 15 years, 16 years, 17 years of age). In some embodiments, the human subject is an adult (e.g., more than 18 years of age). In some embodiments, the human subject is an elderly subject (e.g., more than 60 years of age). In some embodiments, the human subject is 60 years, 65 years, 70 years, 75 years, 80 years, 85 years, 90 years, 95 years, 100 years, or more than 100 years of age.

In some embodiments, the human subject is part of one or more immunologically vulnerable populations. In some embodiments, the human subject is frail (e.g., a subject having frailty syndrome, a malnourished subject, or a subject with a chronic disease causing frailty). In some embodiments, the human subject has a weak immune system, such as an undeveloped (e.g., an infant or a neonate subject), immunosenescent (e.g., an elderly subject), or compromised immune system. Immunosenescent subjects include, without limitation, subjects exhibiting a decline in immune function associated with advanced age. Immunocompromised subjects include, without limitation, subjects with primary immunodeficiency or acquired immunodeficiency such as those suffering from sepsis, HIV infection, and cancers, including those undergoing chemotherapy and/or radiotherapy, as well as subjects to which immunosuppressants are administered, as for organ or tissue transplantation. In some embodiments, the human subject has or is suspected of having one or more disorders or diseases that reduce immune system function and/or increase the risk of infection in the subject by one or more pathogens (e.g., a bacterium, a mycobacterium, a fungus, a virus, a parasite, or a prion). In some embodiments, the human subject is, for example, a subject that has or is suspected of having chronic lung disease, asthma, cardiovascular disease, cancer, a metabolic disorder (e.g., obesity or diabetes mellitus), chronic kidney disease, or liver disease.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: SARS-CoV-2 5′ UTR Mediates Translation Despite the Presence of Nsp1

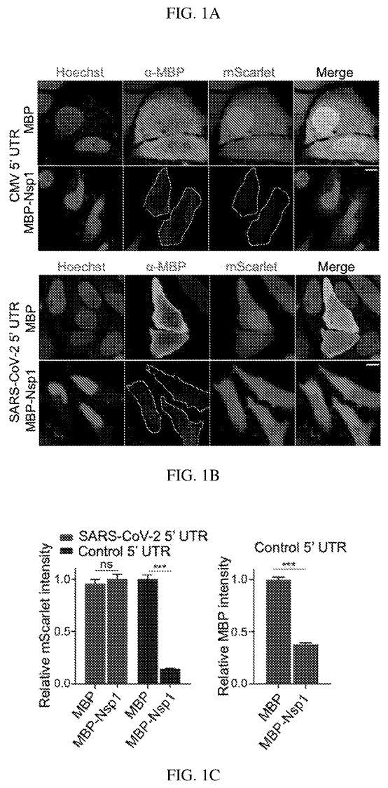

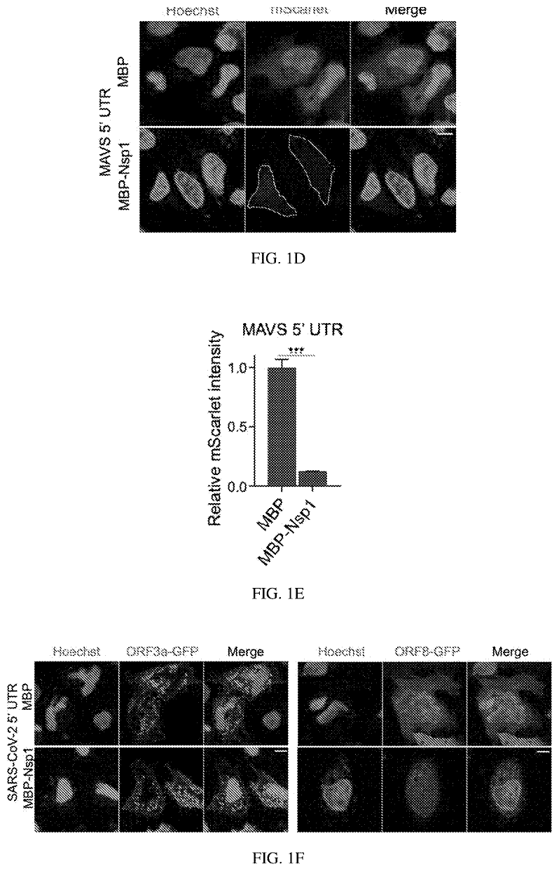

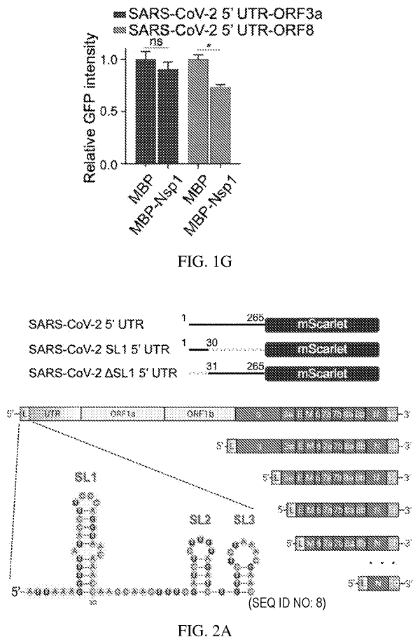

To investigate the function of SARS-CoV-2 non-structural protein 1 (Nsp1) in inhibiting mRNA translation, an mScarlet reporter construct with maltose-binding protein (MBP)-tagged Nsp1 or an MBP control were co-transfected into HeLa cells and mScarlet fluorescence and anti-MBP immunofluorescence was imaged (FIGS. 1A-1B). The mScarlet reporter (hereafter referred to as the “control reporter”) used an expression vector that contains the cytomegalovirus (CMV) promoter and 5′ untranslated region (UTR) and is commonly employed for mammalian cell expression. The MBP and MBP-Nsp1 constructs also used the CMV promoter and 5′ UTR. Upon analysis of the control reporter, mScarlet expression in MBP-Nsp1-transfected cells was found to be reduced by over 7.1-fold (p<0.001) compared to cells co-transfected with MBP-alone (FIGS. 1B-1C).

On the other hand, when the 5′ UTR in the control mScarlet reporter was replaced by the SARS-CoV-2 5′ UTR (referred to as CoV-2 reporter throughout the manuscript), no significant difference in mScarlet expression was observed upon co-expression with MBP-Nsp-1 or MBP, indicating robust evasion of Nsp1-mediated translational suppression (FIGS. 1A-1C). By contrast, in this same experiment, CMV 5′ UTR controlled MBP-Nsp1 showed significantly lower expression than MBP alone (p<0.001) (FIGS. 1B-1C). Thus, expression of Nsp1 from this construct was likely self-limiting, yet still sufficient to inhibit translation of mRNAs with non-SARS-CoV-2 5′ UTR but not the reporter with SARS-CoV-2 5′ UTR. Since the CMV 5′ UTR is not representative of those in human mRNAs, a reporter was also generated containing the 5′ UTR from human mitochondrial antiviral-signaling protein (MAVS), an essential signaling effector responsible for certain virus induced production of type I and III interferons (IFNs), including in response to SARS-CoV-2. This mScarlet reporter was also potently suppressed by MBP-Nsp1, which decreased its expression 8.1-fold relative to MBP alone (p<0.001) (FIGS. 1A, 1D-1E), suggesting that Nsp1-mediated translational suppression of host mRNAs can contribute to disabling critical mediators of the anti-viral IFN response.

To model subgenomic RNAs generated during discontinuous SARS-CoV-2 gene transcription, constructs with SARS-CoV-2 5′ UTR upstream of either green fluorescent protein (GFP) fused to open reading frame (ORF) 3a or ORF8 (ORF3a-GFP or ORF8-GFP, respectively) were tested. ORF3a-GFP expression was not significantly decreased by Nsp1 co-expression, and ORF8-GFP showed only a 1.4-fold decrease (FIGS. 1F-1G), which was modest relative to the 7.1-fold and 8.1-fold decrease observed with CMV 5′ UTR and MAVS 5′ UTR, respectively (FIGS. 1C, 1E). Together these data support that SARS-CoV-2 Nsp1 potently inhibits host protein translation and that SARS-CoV-2 5′ UTR allows evasion of Nsp1-mediated suppression.

### Example 2: The SL1 of the 5′ UTR is Necessary and Sufficient for Evasion of Nsp1-Mediated Translation Suppression

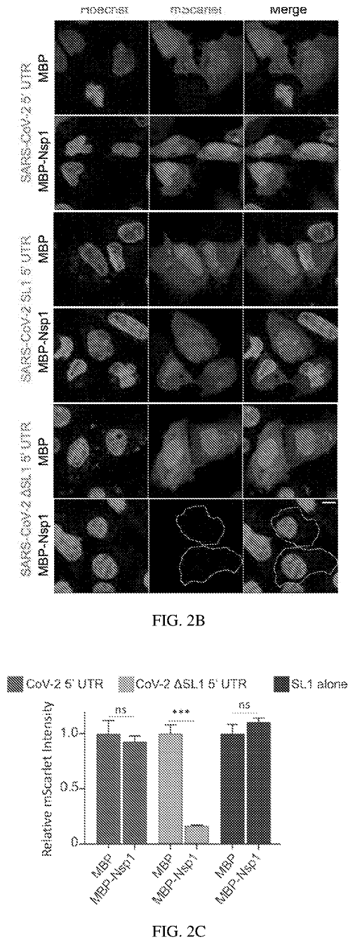

The 5′ UTR of coronaviruses comprises a number of stem-loop structures (FIG. 7), among which stem loop 1 (SL1) has been shown to play critical roles in driving viral replication. This conclusion is also reasonable as the leader sequence driving all SARS-CoV-2 subgenomic RNAs is comprised of just SL1-SL3 instead of the entire 5′ UTR, highlighting the potential importance of SL1 in both viral replication and possibly in promoting evasion from translation suppression by Nsp1. To test the latter function of the SARS-CoV-2 SL1 sequence, 5′ UTR Scarlet reporters were generated without SL1 (ASL1) or with SL1 alone (FIG. 2A). Compared with mScarlet translation in control cells co-transfected with MBP, the translation of SARS-CoV-2 ASL1 5′ UTR mScarlet reporter in MBP-Nsp1 transfected cells was 6.3-fold reduced, similar to the control reporter and MAVS reporter described in Example 1 (p<0.001) (FIGS. 2B-2C). These data suggest that SL1 is completely required for evasion of Nsp1-mediated translation suppression. Interestingly, the reporter bearing only the SL1 sequence in its 5′ UTR was not significantly reduced upon co-expression with Nsp1, indicating that the SL1 sequence is both necessary and sufficient for evasion of Nsp1-mediated translation suppression (FIGS. 2B-2C).

### Example 3: Nsp1-CT and Nsp1-NT are Both Required for Optimal Host Suppression and SL1-Driven Bypass

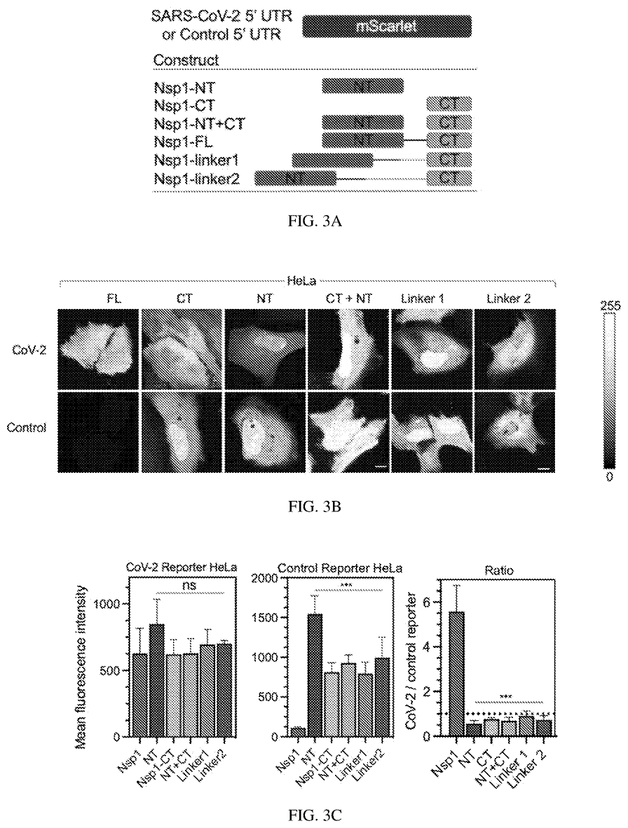

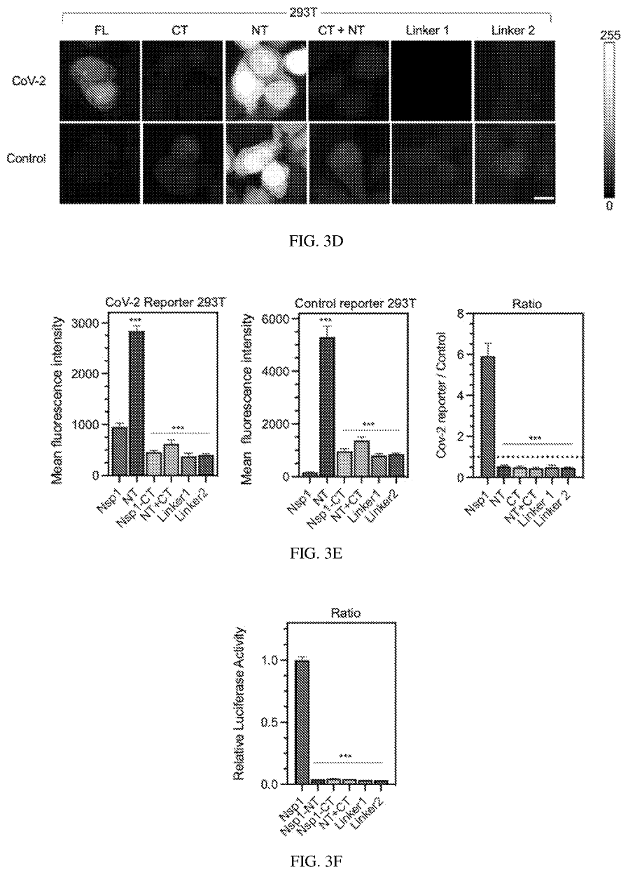

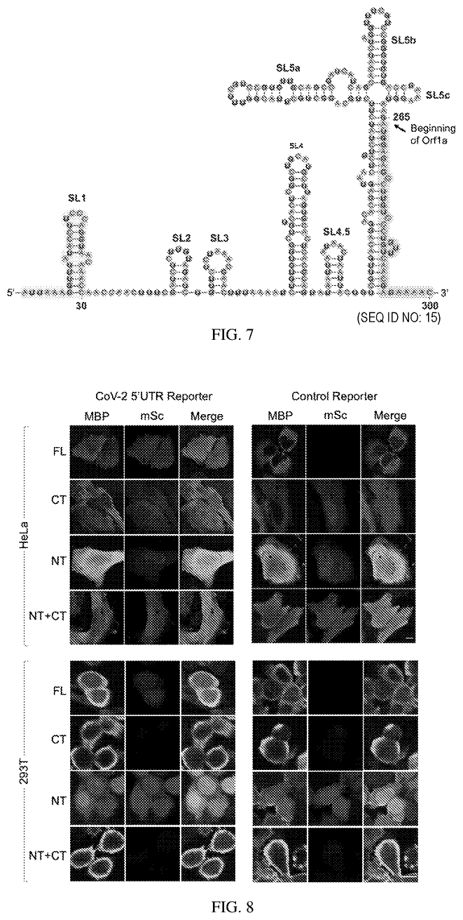

Nsp1 is a 187 amino acid (aa) protein with a 128 aa amino-terminal (NT) domain and a 33 aa carboxy-terminal (CT) domain separated by a short linker region (FIG. 3A). Previous structures of the Nsp1-ribosome complex suggested that Nsp1-CT blocks mRNA entry to the ribosome and should be sufficient to inhibit protein translation. In order to probe the relative functions of these domains in suppressing host translation and allowing bypass by SARS-CoV-2 5′ UTR, HeLa cells were co-transfected with SARS-CoV-2 or control reporters along with full length (FL), NT, CT, or NT+CT Nsp1 constructs (FIGS. 3B-3C). None of these treatments significantly compromised SARS-CoV-2 5′ UTR reporter activity relative to FL Nsp1; however, NT, CT, and NT+CT less efficiently inhibited the control reporter mScarlet fluorescence intensity relative to FL Nsp1, with NT being the least effective (FIGS. 3B-3C, FIG. 8). This experiment was repeated in HEK293T cells and the SARS-CoV-2 5′ UTR reporter activity was significantly higher with co-expression of NT and significantly lower with co-expression of CT in comparison with FL Nsp1, suggesting impaired evasion of CT-imposed translational block (FIGS. 3D-3E, FIG. 8). While FL Nsp1 most effectively suppressed the control reporter, the trend of suppression of the control reporter by NT, CT, or NT+CT mirrored that of the SARS-CoV-2 5′ UTR reporter (FIG. 3E).

These apparently different observations from HeLa versus 293T cell lines were intriguing, but when the ratio of the SARS-CoV-2 5′ UTR reporter to the control reporter fluorescence was examined, a strikingly similar trend was observed. FL Nsp1 co-expression led to a SARS-CoV-2 5′ UTR/control fluorescence ratio of 5.6±1.2 and 5.9±0.62 for HeLa and 293T cells, respectively (FIGS. 3C, 3E). In both cell lines, co-expression of either CT or NT Nsp1 led to a ˜10-fold reduction in SARS-CoV-2 5′ UTR/control fluorescence ratio, and co-expression of NT+CT Nsp1 from separate constructs reduced SARS-CoV-2 5′ UTR/control ratio to a similar extent (FIGS. 3C, 3E). To further validate these observations, a luciferase reporter that also relies on ratiometric normalization to a control reporter was utilized in 293T cells and a similar reduction in SARS-CoV-2 5′ UTR reporter translation selectivity by the different Nsp1 constructs was observed (FIG. 3F). Collectively, these data suggest that the NT and CT of Nsp1 are both important for host translational suppression and that the NT is required for viral evasion of Nsp1-mediated translational suppression. In addition, Nsp1 may be tuned to control the ratio of viral/host translation rather than simply promoting high viral translation, perhaps to maintain some level of host fitness to allow viral replication.

### Example 4: Correct Association and Spacing of NT+CT Via the Nsp1 Linker is Required for Function

The fact that NT+CT expression from different constructs compromised SARS-CoV-2 5′ UTR/control ratio suggested a role for covalent association between the two domains via a linker. To test whether the length of the linker between NT and CT in Nsp1 has any functional effect, an additional 20 residues (linker1) or 40 residues (linker2) were inserted at the Nsp1 linker region (FIG. 3A). Remarkably, the linker extensions dramatically reduced the ratio between SARS-CoV-2 and control reporter expression (p<0.001) in both HeLa and 293T cell lines (FIGS. 3B-3E). These data were also validated in the SARS-CoV-2 5′ UTR luciferase assay (FIG. 3F), suggesting that the NT and CT must somehow cooperate in a spatially specific manner to allow optimal suppression of host translation, and to permit the evasion of suppression on viral translation. A lack of nuclear localization was also observed when visualizing FL, CT, linker1, and linker2 Nsp1. NT alone, however, showed both nuclear and cytosolic localization, suggesting a role for CT and/or linker regions for sequestering Nsp1 in the cytosol (FIG. 8).

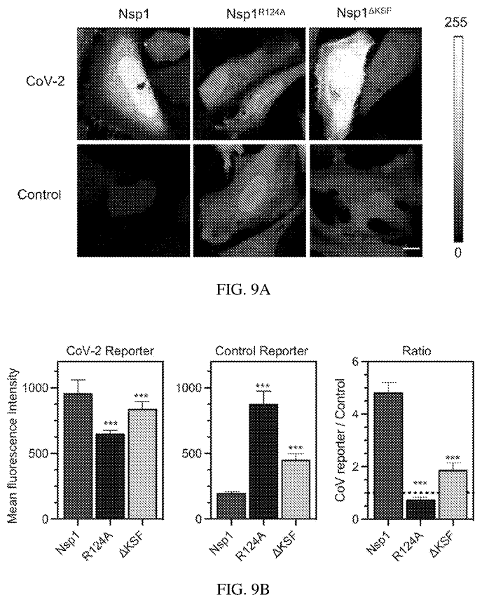

Various naturally occurring mutations have been described in Nsp1 throughout the SARS-CoV-2 pandemic including a 3 amino acid deletion in the Nsp1 linker region (Nsp1ΔKSF) detected in North America and Europe. Given the importance of the Nsp1 linker length in regulating viral to host translation selectivity, the function of this variant was tested. Although Nsp1ΔKSF induced a small significant decrease in SARS-CoV-2 5′ UTR reporter activity, it more than doubled control reporter activity compared to WT Nsp1, and significantly reduced SARS-CoV-2/control translation ratio (p<0.001) (FIGS. 9A-9B). Thus, while lengthening the linker alters regulation of both viral and host translation, the shortened linker in this variant mainly compromised suppression of host translation. Together, these results suggest that the Nsp1 linker length is optimized to coordinate host translational suppression and bypass by SARS-CoV-2 5′ UTR, and that the Nsp1ΔKSF mutant could be less virulent.

It has been recently determined that Nsp1 promotes degradation of host mRNAs whose translation is suppressed, which depends on R124, a key residue in the NT that is conserved in SARS-CoV. To test whether this residue affects host translational shutdown, the control reporter was co-expressed with Nsp1R124A, which increased reporter activity by 5-fold relative to control (p<0.001). Interestingly, this mutation also led to a 1.5-fold decrease in SARS-CoV-2 reporter activity (p<0.001) and significantly reduced the SARS-CoV-2/control ratio (p<0.001; FIGS. 9A-9B). These results suggest that mRNA degradation could indeed contribute to host shutdown and is consistent with the idea that it reduces the pool of available host mRNAs able to compete with SARS-CoV-2 RNA for ribosome association. Other studies have reported that R124A does not interfere with translational suppression of host mRNAs, however these studies relied on in vitro translation in cell extracts which are not optimized to recapitulate Nsp1-directed host mRNA degradation.

### Example 5: SL1 Antisense Oligos (ASOs) Selectively Target SARS-CoV-2 5′ UTR with Nanomolar Potency

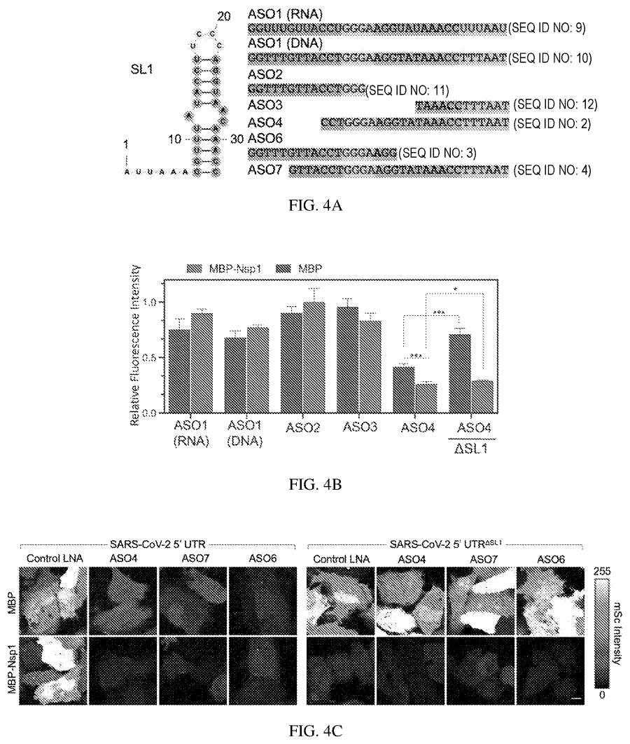

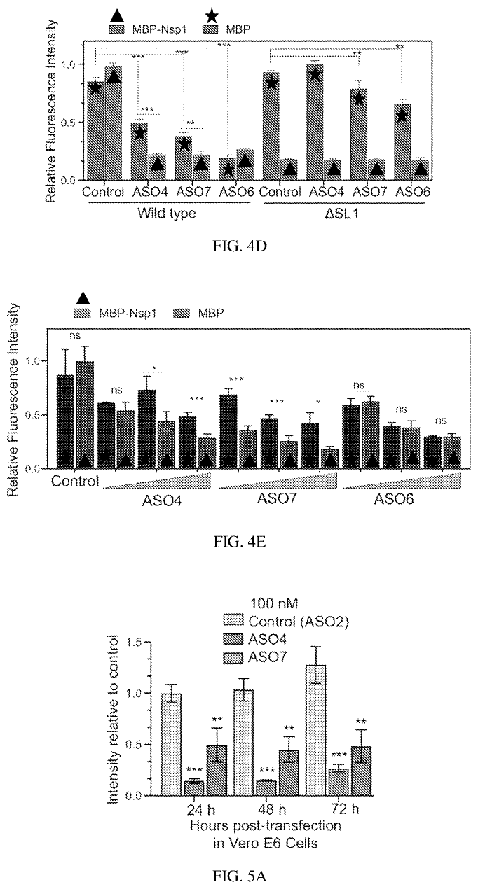

To suppress viral translation, the possibility of disrupting the function of SL1 was examined using anti-sense oligonucleotides (ASO). Since SL1 is sufficient for both viral translation and evasion of Nsp1-mediated translation suppression, ASOs targeting SL1 could represent novel therapeutic opportunities to effectively inhibit viral translation. SL1 starts immediately after the 5′ cap and its structure is dynamically regulated during viral replication. The stem region of SL1 contains 10 Watson Crick base pairs with a bulge at the center (FIG. 4A). Different ASOs were rationally designed to hybridize with various regions of the SL1 and their activity was tested against the SARS-CoV-2 5′ UTR reporter in the presence or absence of Nsp1.

In preliminary experiments, when transfected at 50 nM neither DNA nor RNA anti-SL1 ASOs showed activity against CoV-2 5′ UTR, whether in the presence or absence of Nsp1 (FIG. 4B). Further ASOs were then designed comprising locked nucleic acid (LNA) mixmers targeting various regions of SL1 (FIG. 4A). ASO2 and ASO3 LNAs (short, ≤15 bases) targeting the 5′ and 3′ regions of SL1, respectively, both failed to suppress SL1 activity (FIG. 4B). ASO4, a 24 base LNA against the 3′ region of SL1, successfully suppressed reporter activity on its own when co-transfected at 50 nM with the SARS-CoV-2 reporter and MBP alone (FIG. 4B). Interestingly, this suppression was further enhanced upon co-expression with Nsp1, indicating successful inhibition of SL1-mediated evasion of Nsp1 translational shutdown. In the presence of Nsp1, suppression of SARS-CoV-2 5′ UTR reporter activity by ASO4 was even significantly lower when compared to the ASL1 reporter, demonstrating that ASO4 induces a complete loss of function of the SL1 sequence (FIG. 4B). Two LNA ASOs of similar lengths were additionally designed against the 5′ and 3′ regions of SL1, termed ASO6 and ASO7, respectively. Like ASO4, both ASO6 and ASO7 suppressed the SARS-CoV-2 5′ UTR reporter on their own and showed relatively little activity against the same reporter lacking the SL1 sequence (FIGS. 4C-4D). Interestingly, only ASO4 and ASO7, but not ASO6, synergized with Nsp1 to further suppress SARS-CoV-2 5′ UTR activity. This synergy was consistent over various ASO concentrations (25 nM, 50 nM, and 100 nM) (FIG. 4E). Together, these data suggest that ASO4 and ASO7 suppress viral translation in at least two ways: 1) by causing the SARS-CoV-2 5′ UTR to be less efficient in driving viral translation, and 2) by interfering with the evasion of the SARS-CoV-2 5′ UTR from Nsp-1 mediated suppression.

### Example 6: SL1 ASOs Inhibit SARS-CoV-2 Replication in Vero E6 Cells and can Provide Partial Protection Against SARS-CoV-2-Induced Lethality in K18-hACE2 Mice

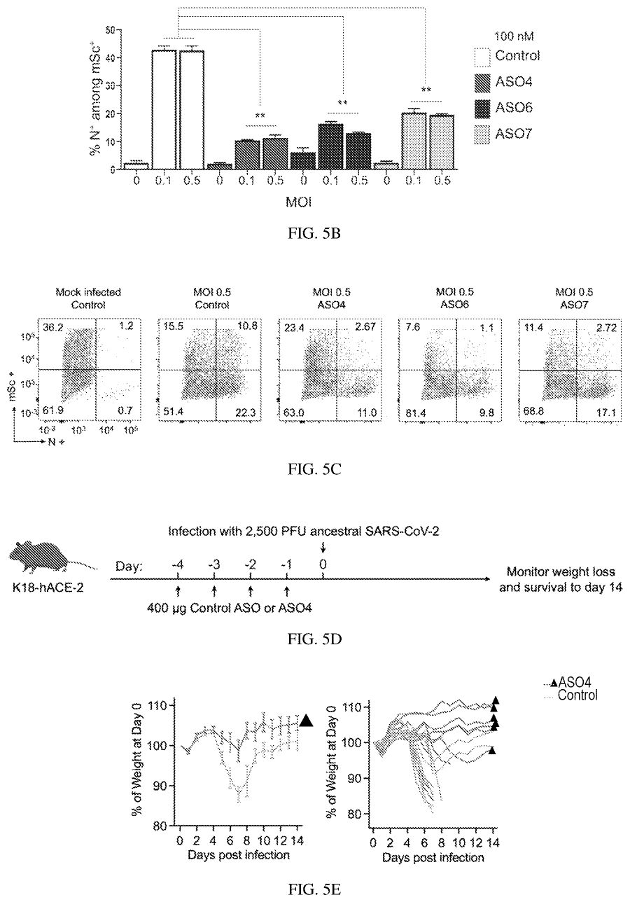

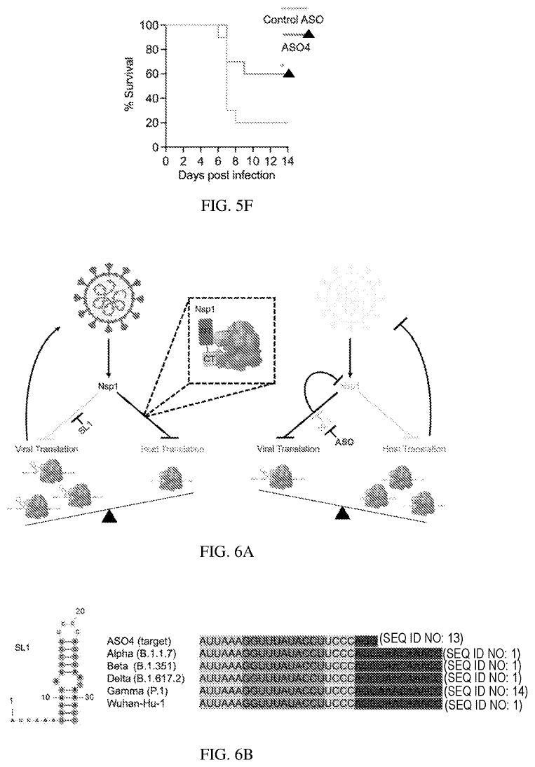

To test whether anti-SL1 ASOs with activity in reporter assays could also inhibit viral replication in Vero E6 cells, the function of these ASOs was first confirmed in the Vero E6 cell line by co-transfecting Vero E6 cells with the SARS-CoV-2 5′ UTR reporter, Nsp1, and various ASOs. When co-transfected at 100 nM, ASO4 and ASO7 caused a 5-fold and 2-fold reduction in reporter activity at 24 hours, respectively (FIG. 5A). Importantly, this reduction in reporter activity persisted for at least 72 hours, indicating that they retained function and stability upon transient delivery to cells (FIG. 5A). Vero E6 cells were then transfected with 100 nM of ASO along with a control mScarlet reporter, followed by SARS-CoV-2 infection at either 0.1 or 0.5 multiplicity of infection (MOI). Cells were fixed 72 hours post-infection and stained for nucleocapsid to mark infected cells (FIG. 5B-5C). Roughly 37% of Vero E6 cells were mScarlet positive when transfected with control ASO and mock infected, indicating transfection efficiency (FIG. 5C). At either MOI 0.1 or 0.5, a ˜4-fold reduction in nucleocapsid and mScarlet positive cells was observed in the presence of AS04, while a ˜3-fold reduction was observed with AS06, and a ˜2-fold reduction was observed with AS07, as compared with a control LNA ASO (FIGS. 5B-5C). These results indicate that ASOs can successfully inhibit SARS-CoV-2 replication in vitro.

Given that ASO4 showed the highest protection against infection in Vero E6 cells, its antiviral efficacy was further tested in vivo using the K18-hACE2 mice expressing the human angiotensin converting enzyme-2 (ACE2), the SARS-CoV-2 entry receptor. This model has been demonstrated to phenotypically recapitulate pathological and clinical features of COVID-19. K18-hACE2 mice were pretreated intranasally with 400 g of naked ASO4 or a control ASO (scrambled) daily for four days before infection with 2,500 plaque forming units (PFU) of ancestral SARS-CoV-2 and monitored for 14 days (FIG. 5D). In the control ASO-treated group, 20% of mice survived infection and exhibited up to of 12% weight reduction on average (FIGS. 5E-5F). The ASO4-treated group showed a significant increase in survival (60%, p=0.0477; log-rank test) with surviving animals showing minimal or no weight loss (FIGS. 5E-5F). These results suggest ASO4 can confer significant protection against SARS-CoV-2 when delivered to the respiratory tract prior to exposure and thus demonstrate the therapeutic efficacy of anti-SL1 targeting in vivo.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. An oligonucleotide comprising a region of complementarity to a severe acute respiratory syndrome coronavirus 2 (SARS-CoV-2) 5′ untranslated region (UTR) target sequence set forth in SEQ ID NO: 1.

2. The oligonucleotide of claim 1, wherein the region of complementarity is at least 17 nucleotides in length.

3. The oligonucleotide of claim 1 or claim 2, wherein the oligonucleotide comprises one more ribonucleosides and/or one or more deoxyribonucleosides.

4. The oligonucleotide of claim 3, wherein at least 40% of nucleotides in the oligonucleotide are ribonucleosides.

5. The oligonucleotide of claim 3, wherein at least 40% of nucleotides in the oligonucleotide are deoxyribonucleosides.

6. The oligonucleotide of any one of claims 3-5, wherein at least one of the one or more ribonucleotides is a modified ribonucleoside.

7. The oligonucleotide of claim 6, wherein the modified ribonucleoside is a locked nucleic acid (LNA)-modified ribonucleoside.

8. The oligonucleotide of claim 7, wherein at least 40% of nucleosides in the oligonucleotide are LNA-modified ribonucleosides.

9. The oligonucleotide of any one of claims 1-8, wherein the oligonucleotide comprises one or more phosphorothioate internucleoside linkages.

10. The oligonucleotide of claim 9, wherein every internucleoside linkage is a phosphorothioate internucleoside linkage.

11. The oligonucleotide of any one of claims 1-10, wherein the oligonucleotide comprises at least 12 consecutive nucleotides of the nucleotide sequence of any one of:
(SEQ ID NO: 2)
CCTGGGAAGGTATAAACCTTTAAT;
(SEQ ID NO: 3)
GGTTTGTTACCTGGGAAGG;
or
(SEQ ID NO: 4)
GTTACCTGGGAAGGTATAAACCTTTAAT.

12. The oligonucleotide of claim 11, wherein the oligonucleotide comprises the nucleotide sequence of any one of:
(SEQ ID NO: 2)
CCTGGGAAGGTATAAACCTTTAAT;
(SEQ ID NO: 3)
GGTTTGTTACCTGGGAAGG;
or
(SEQ ID NO: 4)
GTTACCTGGGAAGGTATAAACCTTTAAT

13. The oligonucleotide of claim 12, wherein the oligonucleotide comprises the nucleotide sequence of any one of:
(SEQ ID NO: 2)
dC+CdTdG+G+GdA+AdGdG+TdA+TdAdA+AdC+CdTdT+TdA+AdT;
(SEQ ID NO: 3)
dG+GdTdT
or
(SEQ ID NO: 4)
dGdT+TdA+C+C+T+G+G+GdAdAdGdG+TdA+TdA+AdA+C+CdTdT+
TdA+AdT,
wherein “dN” represents a deoxyribonucleoside; “+N” represents a locked nucleic acid-modified ribonucleoside; and all nucleotides are linked by phosphodiester internucleoside linkages.

14. The oligonucleotide of any one of claims 1-13, wherein the oligonucleotide is not an oligonucleotide selected from:
(SEQ ID NO: 5)
GAAAGTTGGTTGGTTT;
(SEQ ID NO: 6)
GTTTGTTACCTGGGAA;
or
(SEQ ID NO: 7)
GTTACCTGGGAAGGT.

15. A composition comprising the antisense oligonucleotide of any one of claims 1-14.

16. The composition of claim 15, further comprising a pharmacologically acceptable excipient.

17. The composition of claim 15 or claim 16, further comprising a liposome or a nanoparticle.

18. A method of treating a SARS-CoV-2 infection or reducing the risk of a SARS-CoV-2 infection in a subject in need thereof, the method comprising administering to the subject an effective amount of the antisense oligonucleotide of any one of claims 1-14 or the composition of any one of claims 15-17.

19. The method of claim 18, wherein the administration reduces the translation of SARS-CoV-2 RNA in cells of the subject.

20. The method of claim 18 or claim 19, wherein the administration increases the degradation of SARS-CoV-2 RNA in cells of the subject.

21. The method of any one of claims 18-20, wherein the administration reduces the production of SARS-CoV-2 viral particles in the subject.

22. The method of any one of claims 18-21, wherein the subject is a mammal.

23. The method of any one of claims 18-22, wherein the subject is a human.

24. The method of claim 23, wherein the subject is a human neonate, a human infant, a human adult, or an elderly human.

25. The method of claim 24, wherein the subject is a human adult.

26. The method of claim 24, wherein the subject is an elderly human.

27. The method of claim 26, wherein the administration occurs when the subject is more than 65 years of age.

28. The method of any one of claims 24-27, wherein the subject is immune-senescent, immune-compromised, is infected with human immunodeficiency virus (HIV), has chronic lung disease, asthma, cardiovascular disease, cancer, a metabolic disorder, chronic kidney disease, liver disease, is malnourished, or is frail.

29. The method of any one of claims 18-22, wherein the subject is a companion animal, a research animal, or a domesticated animal.

30. The method of claim 29, wherein the subject is an adult or elderly companion animal.

31. The method of any one of claims 18-30, wherein the administration is systemic.

32. The method of any one of claims 18-31, wherein the administration is intravenous, intramuscular, oral, sublingual, or inhaled.

33. The method of any one of claims 18-32, wherein the administration occurs more than once.

34. The method of any one of claims 18-33, wherein the administration is prophylactic.


SEQUENCE LISTING (15 sequences)

SEQ ID NO: 1 - RNA, 33 nt, Synthetic construct
  source 1..33: mol_type = other RNA
attaaaggtttataccttcccaggtaacaaacc

SEQ ID NO: 2 - DNA, 24 bp, Synthetic construct
  source 1..24: mol_type = other DNA
cctgggaaggtataaacctttaat

SEQ ID NO: 3 - DNA, 19 bp, Synthetic construct
  source 1..19: mol_type = other DNA
ggtttgttacctgggaagg

SEQ ID NO: 4 - DNA, 28 bp, Synthetic construct
  source 1..28: mol_type = other DNA
gttacctgggaaggtataaacctttaat

SEQ ID NO: 5 - DNA, 16 bp, Synthetic construct
  source 1..16: mol_type = other DNA
gaaagttggttggttt

SEQ ID NO: 6 - DNA, 16 bp, Synthetic construct
  source 1..16: mol_type = other DNA
gtttgttacctgggaa

SEQ ID NO: 7 - DNA, 15 bp, Synthetic construct
  source 1..15: mol_type = other DNA
gttacctgggaaggt

SEQ ID NO: 8 - RNA, 74 nt, Synthetic construct
  source 1..74: mol_type = other RNA
attaaaggtttataccttcccaggtaacaaccaaccaactttcgatctcttgtagatctgttctctaaacgaac

SEQ ID NO: 9 - RNA, 33 nt, Synthetic construct
  source 1..33: mol_type = other RNA
ggtttgttacctgggaaggtataaacctttaat

SEQ ID NO: 10 - DNA, 33 bp, Synthetic construct
  source 1..33: mol_type = other DNA
ggtttgttacctgggaaggtataaacctttaat

SEQ ID NO: 11 - DNA, 15 bp, Synthetic construct
  source 1..15: mol_type = other DNA
ggtttgttacctggg

SEQ ID NO: 12 - DNA, 12 bp, Synthetic construct
  source 1..12: mol_type = other DNA
taaacctttaat

SEQ ID NO: 13 - RNA, 25 nt, Synthetic construct
  source 1..25: mol_type = other RNA
attaaaggtttaataccttcccagg

SEQ ID NO: 14 - RNA, 33 nt, Synthetic construct
  source 1..33: mol_type = other RNA
attaaaggtttataccttcccaggaaacaaacc

SEQ ID NO: 15 - RNA, 373 nt, Synthetic construct
  source 1..373: mol_type = other RNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaac